(19) United States
(12) Patent Application Publication
(10) Pub. No.: US 2012/0302941 A1
(43) Pub. Date: Nov. 29, 2012

(54) PHACOEMULSIFICATION SYSTEMS AND ASSOCIATED USER-INTERFACES AND METHODS

(76) Inventors: Dan Teodorescu, Fountain Valley, CA (US); Ahmad Salehi, Irvine, CA (US); Mikhail Boukhny, Laguna Niguel, CA (US)

(21) Appl. No.: 13/113,520

(22) Filed: May 23, 2011

(51) Int. Cl. A61F 9/007; G06F 3/033

(52) U.S. Cl. 604/22; 715/863

(57) ABSTRACT

Phacoemulsification systems and associated user interfaces and methods for use in ophthalmic surgery are disclosed. In some embodiments, a touch screen display of an ophthalmic surgical console provides a graphical user interface that allows a user to both visualize and control various operating parameters of the surgical console and associated subsystems. The controllable operating parameters include, without limitation, aspiration flow rates, IV pole height, vacuum limit pressures, minimum ultrasound power, maximum ultrasound power, on-time, off-time, and/or other values associated with the operating parameters of the ophthalmic surgical console.

### BACKGROUND

The present disclosure relates generally to surgical systems, and, more particularly, to phacoemulsification surgical systems that include graphical user interfaces that allow freeform adjustment of system parameters by a user.

The human eye can suffer a number of maladies causing mild deterioration to complete loss of vision. While contact lenses and eyeglasses can compensate for some ailments, ophthalmic surgery is required for others. Generally, ophthalmic surgery is classified into posterior segment procedures, such as vitreoretinal surgery, and anterior segment procedures, such as cataract surgery. More recently, combined anterior and posterior segment procedures have been developed.

The surgical instrumentation used for ophthalmic surgery can provide a variety of functions depending on the surgical procedure and surgical instrumentation. For example, surgical systems can expedite cataract surgeries (e.g. phacoemulsification procedures) by managing irrigation and aspiration flows into and out of a surgical site, controlling the power supplied to an ultrasound apparatus, and numerous other functions associated with the console.

Modern surgical systems, and in particular, modern ophthalmic surgical systems, are designed to monitor and display multiple parameters of a surgical device or instrument that is connected to the surgical system. In some instances, the surgical device or instrument is controlled by the surgeon through the use of an actuator, such as a foot pedal. Such systems can be complex given the multiple parameters that must be displayed and controlled by a surgeon in the context of an ophthalmic procedure.

Certain known phacoemulsification systems allow for application of ultrasound energy at a fixed level. For example, the foot pedal acts as an on/off switch to activate and deactivate ultrasound energy at a particular power level. When the foot pedal is pressed, the device is activated and the power level is constant or “continuous.” When the foot pedal is subsequently released, the device is deactivated and the ultrasonic energy is switched off.

The original “continuous” power systems were improved by the introduction of a “linear” mode, which allows a surgeon to control power in a variable manner. In “linear” mode, a surgeon controls power based on the foot pedal position so that the power is proportional to or linear with respect to the displacement of the foot pedal. Thus, more power is provided as the surgeon presses the foot pedal, and less power is provided as the foot pedal is released.

Further improvements to the control of phacoemulsification systems involved the introduction of “pulse” and “burst” modes. In “pulse” mode, phacoemulsification energy is provided in periodic pulses at a constant duty cycle. The surgeon increases or decreases the amount of power by pressing or releasing the foot pedal, which increases or decreases the amplitude of the fixed-width pulses. In “burst” mode, power is provided through a series of periodic, fixed width, constant amplitude pulses. Each pulse is followed by an “off’ time. The off-time is varied by the surgeon by pressing and releasing the foot pedal to adjust the power.

In order to accommodate “continuous,” “linear,” “pulse,” and “burst” modes and their operating parameters, known user interfaces typically include several human actionable controllers and fields or elements that occupy particular positions on a display screen. Some known user interfaces include buttons, arrows, switches, bars, and/or knobs for setting desired numeric values of operating characteristics of the surgical system within a limited range of available values. In that regard, certain parameters are fixed or have a constant value regardless of the foot pedal position, whereas other parameters vary, e.g., vary linearly, with the foot pedal position. The user interface is manipulated by a surgeon to provide control signals to the surgical instruments which, in turn, control the modes or types of pulses that are generated in accordance with the inputs of the surgeon into the user interface.

While known user interfaces have been used to perform phacoemulsification procedures in the past, user interfaces for phacoemulsification systems can be improved. Particularly, as described in the present disclosure, the visual and functional aspects of the user interfaces can be enhanced so that surgeons can better define the operating parameters of the system for one or more phases of a procedure and can visualize those operating parameters.

### SUMMARY

The present disclosure provides phacoemulsification systems and associated user interfaces and methods for use in ophthalmic surgery.

In one embodiment, an ophthalmic surgical console system is provided. The system includes an ultrasound generator, a hand piece in communication with the ultrasound generator, a display, and a computing device. The hand piece is electrically energized by the ultrasound generator and a tip of the hand piece is actuated in response to the electrical signals. The display is configured to display an interactive graphical user interface that includes a visualization of an ultrasound operating parameter. The interactive graphical user interface is configured to receive a freeform user input that defines at least a portion of the ultrasound operating parameter. The computing device is in communication with the ultrasound generator and the display. In that regard, the computing device is configured to communicate the ultrasound operating parameter to the ultrasound generator such that the ultrasound signals received by the hand piece from the ultrasound generator are based upon the ultrasound operating parameter as defined by the freeform user input.

In some embodiments, the display is a touch screen and the interactive graphical user interface receives the freeform user input via the touch screen. In that regard, the freeform user input may be a drawing of a function of the ultrasound operating parameter relative to positions of a controller, such as a foot pedal, that is in communication with the computing device. Further, the ultrasound operating parameter may be selected from the group of parameters consisting of ultrasound power level, ultrasound on-time, ultrasound off-time, ultrasound pulses per second, ultrasound duty cycle, and/or other ultrasound parameters.

In some instances, the interactive graphical user interface also includes visualizations of non-ultrasound operating parameters. In that regard, the interactive graphical user interface is configured to receive user inputs that define at least a portion of each of the non-ultrasound operating parameters. The non-ultrasound operating parameters can include one or more of intravenous pole height, aspiration flow rate, vacuum pressure level, vitreous cutter cut rate, vitreous cutter duty cycle, coagulator power level, and/or other non-ultrasound operating parameters. In some embodiments, the interactive graphical user interface is configured to receive a plurality of set points from the user via the touch screen. The set points are utilized to define at least a portion of the non-ultrasound operating parameter. In that regard, the interactive graphical user interface is configured to adjust the visualizations of the non-ultrasound operating parameters based on the set points received from the user. In some instances, the computing device controls the interactive graphical user interface.

In another embodiment, a surgical console system is provided. The system includes a computer system, a touch screen display, a controller movable between a plurality of positions, and a surgical device. The surgical device receives operational signals from the computer system and operates in accordance with the operational signals received from the computer system. The computer system is configured to (1) output an interactive graphical user interface signal to the touch screen display so that the touch screen display can display the interactive graphical user interface, (2) receive freeform user inputs via the touch screen that define values of an operational parameter of the surgical device in relation to the plurality of positions of the controller, and (3) send the operational signals to the surgical device based on the position of the controller and the values of the operational parameter as defined by the freeform user input.

In some instances, the surgical device is an ultrasound hand piece and the operating parameter is longitudinal ultrasound power. The computer system may be further configured to receive a second user input via the touch screen that defines values of an additional operational parameter of the surgical device in relation to the plurality of positions of the controller and send the operational signals to the surgical device based on the position of the controller and the values of the operational parameters. In some instances, the additional operating parameter is a torsional ultrasound power. In other instances, the surgical device is a vitreous cutter. In that regard, the operating parameter can be vitreous cut rate and/or vitreous duty cycle.

In another embodiment, an ophthalmic surgical method is provided. The method includes receiving, via an interactive graphical user interface, a freeform input from a user of an ophthalmic surgical system. The freeform input defines a characteristic of an operating parameter of the ophthalmic surgical console. The method also includes operating the ophthalmic surgical console such that the operating parameter is controlled in accordance with the freeform input received from the user. In some instances, the freeform user input received from the user is a drawing of a function of the operating parameter of the ophthalmic surgical system relative to positions of a controller of the ophthalmic surgical system. In that regard, the operating parameter may be one or more of intravenous pole height, aspiration flow rate, vacuum limit pressure, ultrasound power, ultrasound on-time, ultrasound off-time, and/or other operating parameters.

Other aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a front view of an ophthalmic surgical console 100 according to one embodiment of the present disclosure.

FIG. 2 is a block diagram of the ophthalmic surgical console 100 of FIG. 1.

FIG. 3 is a portion of an interactive graphical user interface (“GUI”) visually illustrating select operating parameters of the ophthalmic surgical console according to one embodiment of the present disclosure.

FIG. 4 is a graph illustrating ultrasonic power versus time according to one embodiment of the present disclosure.

FIG. 5 is a graph illustrating hand piece tip position versus time in accordance with the ultrasonic power graph of FIG. 4.

FIG. 6 illustrates a pair of graphs illustrating longitudinal power versus time and torsional power versus time according to one embodiment of the present disclosure.

FIG. 7 is a portion of an interactive graphical user interface (“GUI”) that allows a user to define ultrasound power versus time using freeform input according to one embodiment of the present disclosure.

FIGS. 8-10 illustrate adjustment of an operating parameter using linear interpolation according to one embodiment of the present disclosure. In that regard, FIG. 8 illustrates a baseline or original graphical representation for the operating parameter. FIG. 9 illustrates a plurality of user-selected set points relative to the original graphical representation of the operating parameter shown in FIG. 8. FIG. 10 illustrates a modified graphical representation of the operating parameter in accordance with the user-selected set points of FIG. 9.

FIGS. 11 and 12 illustrate adjustment of an operating parameter using a smooth curve interpolation according to one embodiment of the present disclosure. In that regard, FIG. 11 illustrates a plurality of user-selected set points relative to the original graphical representation of the operating parameter shown in FIG. 8. FIG. 12 illustrates a modified graphical representation of the operating parameter in accordance with the user-selected set points of FIG. 11.

FIGS. 13-16 illustrate adjustment of an operating parameter using a freeform user input according to one embodiment of the present disclosure. In that regard, FIG. 13 illustrates a first portion of a freeform user input relative to the original graphical representation of the operating parameter shown in FIG. 8. FIG. 14 illustrates a second portion of the freeform user input along with the first portion of FIG. 13 relative to the original graphical representation of the operating parameter shown in FIG. 8. FIG. 15 illustrates a third portion of the freeform user input along with the first and second portions of FIGS. 13 and 14 relative to the original graphical representation of the operating parameter shown in FIG. 8. FIG. 16 illustrates a modified graphical representation of the operating parameter in accordance with the freeform user inputs of FIGS. 13-15.

FIG. 17 illustrates adjustment of an operating parameter using a combination of linear interpolation, smooth curve interpolation, and freeform user input according to one embodiment of the present disclosure.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the disclosure is intended. Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one embodiment may be combined with the features, components, and/or steps described with respect to other embodiments of the present disclosure.

Embodiments of the present disclosure are directed to a graphical user interface that provides improved control over the operating parameters of an ophthalmic surgical system and visualization of those parameters. Representations of values, characteristics, and/or functions of the operating parameters are displayed visually as part of a graphical user interface on a touch screen of the ophthalmic surgical system. In some embodiments, the visual representations (and the corresponding operating parameters associated therewith) can be changed by a user touching a display screen and modifying the visual representation of the operating parameter. In some instances, the user inputs set points that the system utilizes to define the operating parameter. In other instances, the user draws the visualization of the operating parameter in freeform. In yet other instances, the user uses a combination of set points and freeform drawing to define an operating parameter. As discussed below, a wide variety of operating parameters of a phacoemulsification system are controllable in this manner.

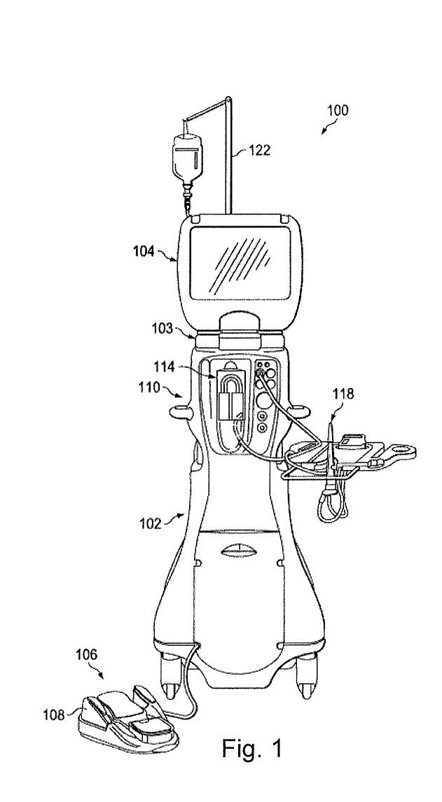

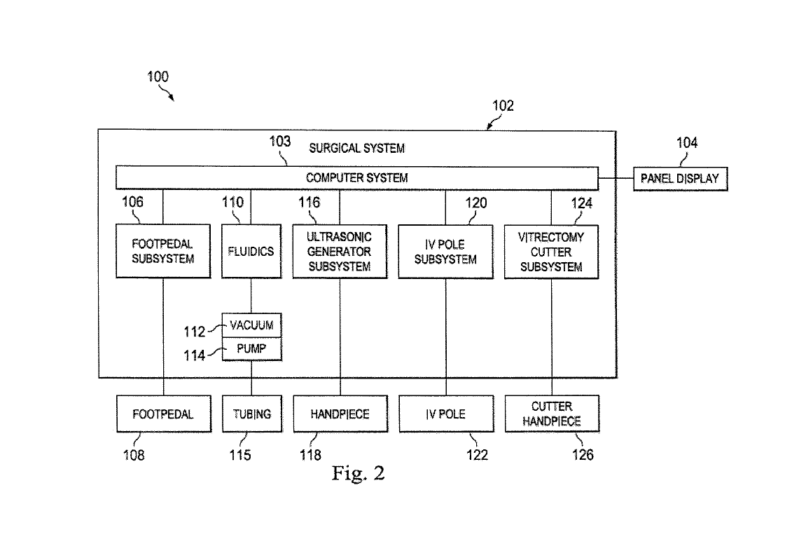

FIG. 1 illustrates an ophthalmic surgical console, generally designated 100, according to an exemplary embodiment of the present disclosure. FIG. 2 is a block diagram of the console 100. The console 100 includes a base housing 102 with a computer unit 103 and an associated display screen 104 showing data relating to system operation and performance during ophthalmic procedures, such as a phacoemulsification procedure. The console also includes a number of subsystems that are used together to perform the procedures. For example, the subsystems include a foot pedal subsystem 106 including, for example, a foot pedal 108, a fluidics subsystem 110 including an aspiration vacuum 112 and an irrigation pump 114 that connect to tubing 115, an ultrasonic generator subsystem 116 including an ultrasonic oscillation hand piece 118, an intravenous (IV) pole subsystem 120 including a motorized IV pole 122, and a pneumatic vitrectomy cutter subsystem 124 including a vitrectomy hand piece 126. To optimize performance of the different subsystems during surgery, the operating parameters differ according to, for example, the particular procedure being performed, the different stages of the procedure, the surgeon's personal preferences, whether the procedure is being performed in the anterior or posterior portion of the patient's eye, and so on.

The different subsystems in the base housing 102 comprise control circuits for the operation and control of the respective microsurgical instruments. The computer system 103 governs the interaction and relationship between the different subsystems to properly perform an emulsification surgical procedure. To do this, the computer system 103 includes a processor and memory and is programmed with instructions for controlling the subsystems to carry out an ophthalmic procedure. In some aspects, the user interfaces of the present disclosure facilitate customization of the operating parameters of the subsystems. In that regard, the customization of the operating parameters is reflected in corresponding modifications to the programmed instructions for controlling the subsystems utilized by the computer system 103.

As shown in FIG. 1, the display screen 104 rests on the base housing 102 for viewing and access by an operator. In some instances, the display screen is part of a swivel monitor that can be positioned in a variety of orientations such that the display screen 104 is conveniently positioned for whoever needs to see it. In that regard, swivel monitor 110 can swing from side to side, as well as rotate and tilt. As will be discussed in detail below, the display screen 104 provides a graphical user interface (“GUI”) that allows a user to interact with the ophthalmic surgical console 100 to control and/or define various operating parameters of the ophthalmic surgical console.

An input device permits a user to control aspects of the console 100 through the display 104. In this embodiment, the input device is a touch screen device responsive to selections made directly on the display 104. However, other input devices, such as a standard computer keyboard, a standard pointing device (e.g., a mouse or trackball), or other input device are used in combination with or in lieu of the touch screen in some instances. In the exemplary embodiments described herein, the display screen 104 is a touch screen that shows an interactive graphical user interface that permits surgeons, scientists, medical personnel, and/or other users to select, adjust, define, and/or visualize the operating parameters of the different subsystems of the console 100. Accordingly, a user may change or adjust the operational parameters of the different subsystems and/or the relationships between the operational parameters of the different subsystems from the default settings of the console 100.

The ophthalmic surgical console 100 is provided by way of example and embodiments of the present disclosure can be implemented with a variety of surgical systems. Examples of ophthalmic surgical systems in which embodiments of the present disclosure can be implemented include, for example, the Infiniti® Vision System surgical system available from Alcon Laboratories Inc. of Fort Worth, Tex. Persons skilled in the art will appreciate that the embodiments described below can be utilized with other types of surgical equipment including, but not limited to, neurosurgery equipment, where control of various instruments is also performed with a remote actuator, such as a foot pedal. In general, embodiments of the present disclosure can be utilized with any surgical console that has a touch screen and controls multiple operating parameters. However, for purposes of explanation, not limitation, the remainder of this specification describes embodiments related to phacoemulsification procedures and their associated operating parameters.

Still referring to FIG. 1, a graphical user interface (GUI) is displayed on touch screen display 104 such that a user is able to interact and control aspects of the ophthalmic surgical console 100 through interaction with the GUI. For example, the user may control various operating parameters associated with vitreous cutting, vacuum extraction, scissors, fluidic control, ultrasonic lens removal, and/or other functions of the ophthalmic surgical console 100. In that regard, the user may define or set values associated with these exemplary parameters, including but not limited to aspiration flow rates, IV pole height, vacuum limit pressures, minimum power, maximum power, on-time, off-time, and/or other values associated with the operating parameters of the ophthalmic surgical console 100. Further, the user may define or set the values separately for different stages of an ophthalmic procedure. One or more visual representations of the operating parameters and/or the associated values are displayed on the touch screen 104 for the user's viewing.

The visual representations can be programmed, monitored, and manipulated by a user. In that regard, the visual representations can be adjusted (as discussed in greater detail below) to customize control over the operation of surgical devices or subsystems associated with the ophthalmic surgical console 100 and to provide specific operating parameter values or ranges of values during different stages of the procedure based on inputs into a controller, such as foot pedal 108, of the ophthalmic surgical system 100. For example, the values and/or functions of the operating parameters can be defined to change as a position of the controller, such as depression of the foot pedal 108, is changed. In that regard, the system will invoke the programmed set of operating parameters and associated values that appear on the display screen to control the attached surgical devices, components, and/or subsystems in response to the changing position of the controller.

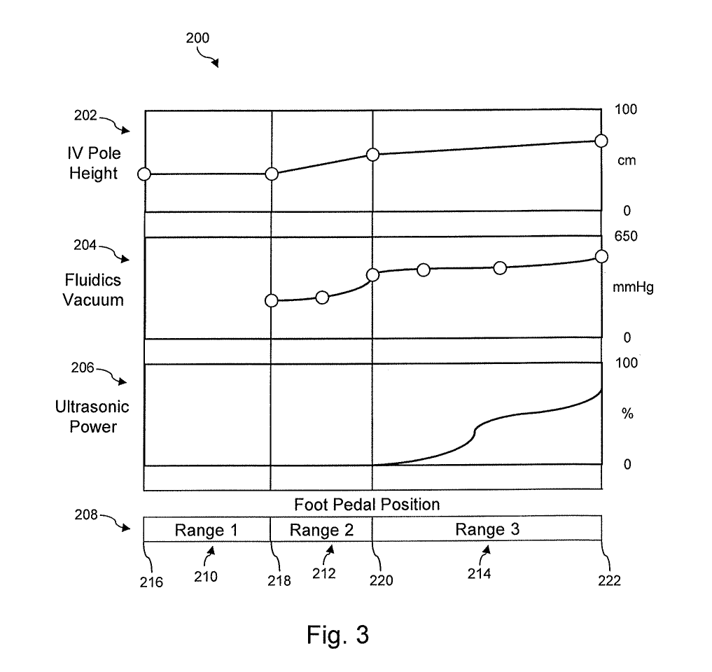

Referring now to FIG. 3, shown therein is a portion of an interactive graphical user interface (“GUI”) 200 according to one embodiment of the present disclosure. As shown, the GUI 200 includes visual representations of three operating parameters relative to various controller positions. More specifically, the GUI 200 includes a graph 202 of intravenous pole height, a graph 204 of fluidics vacuum pressure, and a graph 206 of ultrasonic power each displayed relative to various ranges of foot pedal position as indicated in scale 208. In the illustrated embodiment, the foot pedal position has been divided into three ranges 210, 212, and 214, which may also be referred to as range 1, range 2, and range 3, respectively. The ranges 210, 212, and 214 are defined by vertical dividers or boundary lines 216, 218, 220, and 222. In that regard, the ranges 210, 212, and 214 generally correspond to the amount of depression of the foot pedal 108. Accordingly, in some instances boundary line 216 corresponds to no foot pedal depression (i.e., no actuation of the foot pedal), while boundary line 222 corresponds to 100% foot pedal depression (i.e., full actuation of the foot pedal). When the foot pedal 108 is depressed so that it falls within a particular range, the surgical console 100 operates the subsystems in accordance with the operating parameters and parameter values defined for that particular stage, as reflected on the display screen.

As shown in FIG. 3, boundary lines 216, 218, and 220 generally define transitions between different stages of an ophthalmic procedure. In that regard, often different stages of a surgical procedure require control over different sets of the parameters. For example, some surgical stages will contain ultrasound parameters along with the fluidics parameters, flow and vacuum limit, while other stages will contain only fluidics parameters, while yet other steps will not contain either ultrasound or fluidics parameters (e.g., a coagulation surgical stage where only a coagulation power parameter is included). In some embodiments, the first stage controls only IV pole height, the second stage adds fluidics parameters (e.g., flow rate and/or vacuum/pressure level), vitrectomy cutter parameters (e.g., cut rate, duty cycle), and coagulator parameters (e.g., power level), and the third stage adds ultrasound parameters (e.g., power, longitudinal power, torsional power, on-time, off-time).

In the illustrated embodiment of FIG. 3, boundary line 216 marks the beginning of a first stage of an ophthalmic procedure as it represents the start of controller actuation (i.e., foot pedal depression). In that regard, the first stage of the procedure (corresponding to range 210 of foot pedal position) contains only the IV pole height parameter, as both the fluidics vacuum pressure and the ultrasonic power are set to zero. Boundary line 218 marks the end of the first stage and the beginning of a second stage of the ophthalmic procedure (corresponding to range 212 of foot pedal position) where the fluidics vacuum pressure parameter is added to the IV pole height parameter, but the ultrasonic power is still set to zero. Finally, boundary line 220 marks the end of the second stage and the beginning of a third stage of the procedure (corresponding to range 214 of foot pedal position) where the ultrasonic power parameter is added to the IV pole height and fluidics vacuum pressure parameters.

It is understood that the number of stages and combinations of parameters for these stages is for explanation and not limitation. It is understood that the present disclosure is applicable to ophthalmic procedures having any number of stages (from 1 stage to more than 10 stages) and that any combination of operating parameters is controllable, if desired, at any stage of the ophthalmic procedure. Accordingly, the present disclosure should be understood to include control of any and all possible combinations of operating parameters at any and all ranges of controller positions or stages or an ophthalmic procedure. However, for the sake of clarity and simplicity the following discussion will focus on the exemplary operating parameter combinations and foot pedal positions illustrated in FIG. 3. In that regard, although the discussion below describes irrigation, aspiration, vacuum, and power parameters, persons skilled in the art will appreciate that other surgical procedures and other phacoemulsification systems involve other parameters. Accordingly, the exemplary operating parameters described below in the context of a phacoemulsification are not limiting, but explanatory as other operating parameters are understood to be within the scope of the present disclosure.

Referring to FIG. 3, as the foot pedal is initially depressed the foot pedal will move from boundary line 216 (representing no depression of the foot pedal) into range 210 or stage 1 of the ophthalmic procedure. During stage 1, irrigation fluid is supplied to the surgical site in accordance with the value defined in the graph 202 representing the IV pole height. A source of irrigation can be an elevated bottle or a bag that includes Balanced Salt Solution (BSS) or saline attached to the IV pole 122 of the ophthalmic surgical console 100. In some instances, BSS is delivered to the site by opening a valve allowing the BSS to flow toward the surgical site. In the illustrated embodiment, the graph 202 indicates that the height of the IV pole 122 is to be held constant during stage 1 of the ophthalmic procedure by representing the IV pole height as a horizontal line.

As the foot pedal is depressed further, the foot pedal position will move through range 210 and pass boundary line 218 into range 212, which corresponds to stage 2 of the ophthalmic procedure. In stage 2, aspiration is initiated by activating a peristaltic pump. Thus, following the start of irrigation in stage 1, aspiration is added in stage 2. In that regard, during stage 2, irrigation fluid is supplied to the surgical site in accordance with the value defined in the graph 202 representing the IV pole height, while aspiration is supplied in accordance with the value defined in graph 204 representing the vacuum pressure. The graph 202 indicates that the height of the IV pole 122 is to be increased linearly during stage 2 of the ophthalmic procedure by representing the IV pole height as a straight line with a constant slope between boundary lines 218 and 220. The graph 204 indicates that the vacuum pressure is to be increased non-linearly during stage 2 of the ophthalmic procedure. As shown, the vacuum pressure is depicted by curved line segments extending between boundary lines 218 and 220. In some instances, the curved line segments are defined by an exponential, a polynomial, a smooth curve or best fit interpolation, or a user-defined freeform input.

As the foot pedal is depressed further, the foot pedal position will move through range 212 and pass boundary line 220 into range 214, which corresponds to stage 3 of the ophthalmic procedure. In stage 3, ultrasound power is initiated. Thus, following the start of irrigation and aspiration in stages 1 and 2, ultrasound power is added in stage 3. Accordingly, during stage 3 irrigation, aspiration, and ultrasound power are all controlled. In that regard, irrigation fluid is supplied to the surgical site in accordance with the values defined in the graph 202 representing the IV pole height, aspiration is supplied in accordance with the values defined in graph 204 representing the vacuum pressure, and ultrasound power is supplied to the hand piece in accordance with the values defined in graph 206 representing the ultrasound power. Again, the graph 202 indicates that the height of the IV pole 122 is to be increased linearly during stage 3 of the ophthalmic procedure by representing the IV pole height as a straight line with a constant slope between boundary lines 220 and 222. However, as shown, the linear increase of IV pole height is less in stage 3 than in stage 2. The graph 204 indicates that the vacuum pressure is to be increased non-linearly during stage 3 of the ophthalmic procedure. Similar to stage 2, the vacuum pressure is again depicted by curved line segments extending between boundary lines 220 and 222 that may be defined by an exponential, a polynomial, or a smooth curve or best fit interpolation. The graph 206 indicates that the ultrasound power is to be increased non-linearly during stage 3 of the ophthalmic procedure by representing the ultrasound power as curved line segments between boundary lines 220 and 222. In some instances, the curved line segments of the ultrasound power are defined by an exponential, a polynomial, a smooth curve or best fit interpolation, or a user-defined freeform input. In the illustrated embodiment, the graph 206 is based upon a user-defined freeform input, which will be discussed in greater detail below.

Releasing or raising the foot pedal results in the opposite sequence deactivating ultrasound power, deactivating aspiration, and then deactivating irrigation. Accordingly, the surgeon or other user can activate or de-activate the various operating parameters during the ophthalmic procedure by depressing and releasing the foot pedal as needed to reach the desired foot pedal position and related operating parameters values.

The operating parameters of the surgical device during the various stages of the ophthalmic procedure are dictated by the information represented in the graphical user interface shown on touch screen display 104 and programmed into the computer system 103 of the console 100. Accordingly, exemplary ways in which the operating parameters, such as irrigation flow, aspiration rate, vacuum level, and ultrasound power, are displayed and adjusted is described below in further detail. Persons skilled in the art will appreciate that the same representation and adjustment techniques can also be applied to other parameters, during other stages of an ophthalmic procedure, and during other surgical procedures.

At least some of the values or properties associated with the ultrasound component of a phacoemulsification procedure are defined via the graphical user interface displayed on the touch screen display 104. In that regard, the application of periodic ultrasound pulses in the context of phacoemulsification procedures can generally be described based on power, the duration of the pulses, the “On” or active time, and the duration of “Off” time or the duration between pulses. Alternatively, the ultrasound pulses can be specified using pulse rate and duty cycle. The pulse rate is the number of pulses contained in unit time, while the duty cycle is the portion of the ultrasound cycle when the ultrasound is active. In other words, the duty cycle can be defined as On Time/(On Time+Off Time).

The graphical user interfaces of the present disclosure provide the user with improved control over the ultrasound driving or pulse modes that are generated by a phacoemulsification surgical system and improved control over the operating parameters associated with the different pulse modes. In that regard, embodiments of the graphical user interfaces provide display elements that can be quickly and easily adjusted by a surgeon to customize the various pulse modes. The pulse modes that can be selected include “continuous,” “pulse,” and “burst” modes, as well as hybrid or combinations of these modes. In that regard, visual representations of the operating parameters, characteristics, and/or functions of pulses are displayed on the display 104. The visual representations, and thereby the corresponding operating parameters, characteristics, and/or functions, can be changed by interfacing with the display screen as discussed below. In some instances, a separate window (e.g., a pop up window) can be generated in response to touching the display screen. The visual representations and/or values of the corresponding operating parameters, characteristics, and/or functions, can be changed within the separate window. In other instances, a separate window is not generated and adjustments are made in the existing window of the graphical user interface.

Embodiments of the present disclosure provide improvements over known interfaces by allowing power, on-time, off-time, and other pulse parameters to be defined to increase linearly, increase non-linearly, decrease linearly, decrease non-linearly, and remain substantially constant relative to displacement of a foot pedal. These settings are depicted visually to a user such that the user can easily see whether the power, on-time, and/or off-time is to decrease or increase linearly, decrease or increase non-linearly, or remain constant for a particular stage of a procedure. In that regard, different pulse modes can be generated by selecting the manner in which the power, on-time, and the off-time vary (or not vary).

In some instances, the ultrasound operating parameter values are selected to provide continuous power. In that regard, the off-time can be set to zero by the user. Accordingly, the power is off for “0” time (i.e., the power is on all of the time) and is, therefore, continuous. In a continuous power mode, the on-time representation is constant or fixed. Since the power is continuous, any non-zero “on-time” value supported by the system can be used.

In other instances, the ultrasound operating parameter values are selected to provide what is commonly referred to as “pulse” mode. In “pulse” mode, the ultrasound power is provided in periodic pulses at a constant duty cycle. In that regard, both the on-time and the off-time are set to a constant, non-zero value. For example, the on-time can be set to 25 ms and the off-time set to 100 ms. This will provide 8 pulses per second and the ratio of the ultrasound on-time to the total cycle time is 25/125=0.2, or a duty cycle of 20%. Accordingly, the duty cycle of the “pulse mode” can be adjusted by adjusting the ultrasound on-time and/or the off-time.

In yet other instances, the ultrasound operating parameter values are selected to provide what is commonly referred to as “burst” mode. In “burst” mode, the ultrasound power is provided with a constant on-time, but a varying off-time. In some instances the off-time decreases with foot pedal displacement. Accordingly, in such instances, the duty cycle increases with foot pedal displacement. For example, the on-time can be set to a constant 50 ms, while the off-time is set to decrease linearly from 2500 ms to 0 as the foot pedal is depressed. The result is that when the foot pedal is pushed all the way down the ultrasound power is continuous because the off-time reaches 0.

In still other instances, the ultrasound operating parameter values are selected to provide a varying on-time, but a constant off-time. In some instances the on-time decreases with foot pedal displacement. Accordingly, in such instances, the duty cycle increases with foot pedal displacement. For example, the on-time can decrease from 150 ms to 30 ms as the foot pedal is depressed, while the off-time is maintained constant at 20 ms. The result is that this type of ultrasound parameter profile can be “adaptive” to various lens hardnesses. For example, typically when the surgeon sees that a given foot pedal depression is not resulting in the desired rate of lens removal, the surgeon will press the foot pedal down further. The greater power typically associated with depressing the foot pedal further also results in increased repulsion. However, repulsion is reduced, minimized, or eliminated by the present ultrasound operating parameter profiles because the duration of the ultrasound pulse (i.e., the on-time) is shortened with the increased power associated with depressing the foot pedal further. This ultrasound operating parameter profile can be particularly useful when a user is attempting to extract extremely mature cataracts, which are more prone to repulsion at higher powers due to increased hardness.

With respect to defining the ultrasound operating parameter profiles, Initial, minimum, and/or maximum values of the power, on-time, and off-time can be set or programmed by the user in some instances. The system can be configured so that the minimum power value is 0% or another desired value when the foot pedal is released (i.e., the foot pedal is not depressed). Also, the initial on-time or, alternatively, the minimum on-time, can be 0 ms or another desired value. Similarly, the initial off-time or, alternatively, the minimum off-time, can be 0 ms or another desired value.

For simplicity, the following discussion will focus on ultrasound power, but it is understood that the same concepts for visually representing and modifying the ultrasound power parameter are also applicable to the other operating parameters associated with defining the ultrasound pulses as well as the non-ultrasound operating parameters of the ophthalmic surgical console 100 and associated subsystems. For example and without limitation, the concepts are also applicable to flow rates, IV pole heights, aspiration rates, vacuum pressures, ultrasound on-times, ultrasound off-times, ultrasound power change rates, ultrasound pulses per second, ultrasound duty cycles, vitreous cutter cut rate, vitreous cutter duty cycle, coagulator power levels, and/or other operating parameters associated with phacoemulsification procedures.

Generally, the visual representation of ultrasound power shown on the display can have various shapes depending on the desired relationship or function of the ultrasound power to the position of the foot pedal. The visual representation of the ultrasound power can be linear, non-linear, and/or combinations thereof relative to the foot pedal position in order to represent a corresponding linear, non-linear, and/or combined linear and non-linear function of the power with respect to controller position. A linear representation can be an increasing linear representation (i.e., a straight line having a constant, positive slope), a horizontal or constant linear representation (i.e., a straight line having a constant, zero slope), a decreasing linear representation (i.e., a straight line having a constant, negative slope), and combinations thereof. A non-linear representation can be an increasing non-linear representation, a decreasing non-linear representation, and combinations thereof. Exemplary non-linear representations include exponential, polynomial, user-defined freeform representations, and/or combinations thereof.

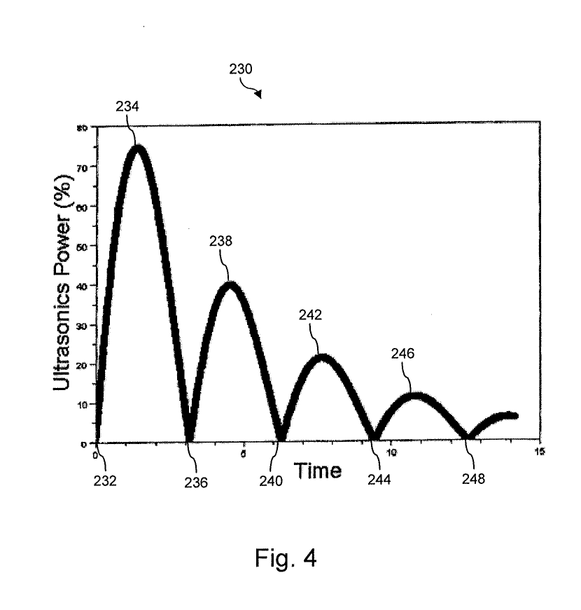

Referring now to FIG. 4, shown therein is a graph 230 mapping ultrasonic power relative to time according to an exemplary embodiment of the present disclosure. As shown, the ultrasonic power varies non-linearly with respect to time. In that regard, the ultrasonic power repeatedly increases and decreases with time where the corresponding maximum power of each increase in power is less with each cycle. As shown, the ultrasound power starts at point 232 with a value of zero, increases to peak 234 (where it reaches approximately 75% of maximum power), decreases back to zero at point 236, increases to peak 238 (where it reaches approximately 40% of maximum power), decreases back to zero at point 240, increases to peak 242 (where it reaches approximately 20% of maximum power), decreases back to zero at point 244, increases to peak 246 (where it reaches approximately 10% of maximum power), decreases back to zero at point 248, and increases from there to where the graph 230 stops. When implemented during a surgical procedure, this ultrasound power profile will cause a corresponding displacement of the hand piece tip by producing oscillations of varying amplitude as driven by the defined ultrasound power.

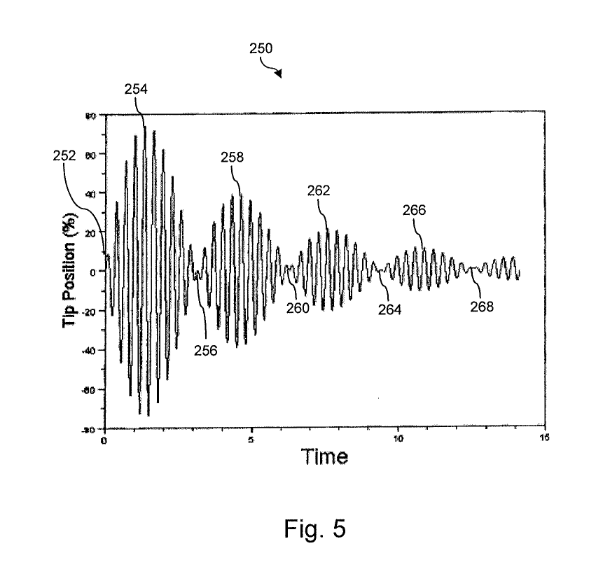

Referring now to FIG. 5, shown therein is a graph illustrating hand piece tip position, as a percentage of maximum tip displacement, versus time in accordance with the ultrasonic power graph 230 of FIG. 4. As shown the hand piece tip position generally tracks the profile of the ultrasound power profile defined by the graph 230. More specifically, the increases and decreases in tip displacement correspond directly to the increases and decreases in ultrasound power. In that regard, the hand piece tip starts at point 252 with a displacement of zero, increases to peak 254 (where it reaches approximately 75% of maximum displacement), decreases back to approximately zero at point 256, increases to peak 258 (where it reaches approximately 40% of maximum displacement), decreases back to approximately zero at point 260, increases to peak 262 (where it reaches approximately 20% of maximum displacement), decreases back to approximately zero at point 264, increases to peak 266 (where it reaches approximately 10% of maximum displacement), decreases back to approximately zero at point 268, and increases from there to where the graph 250 stops. When implemented during a surgical procedure, this ultrasound power profile will cause a corresponding displacement of the hand piece tip by producing oscillations of varying amplitude as driven by the defined ultrasound power.

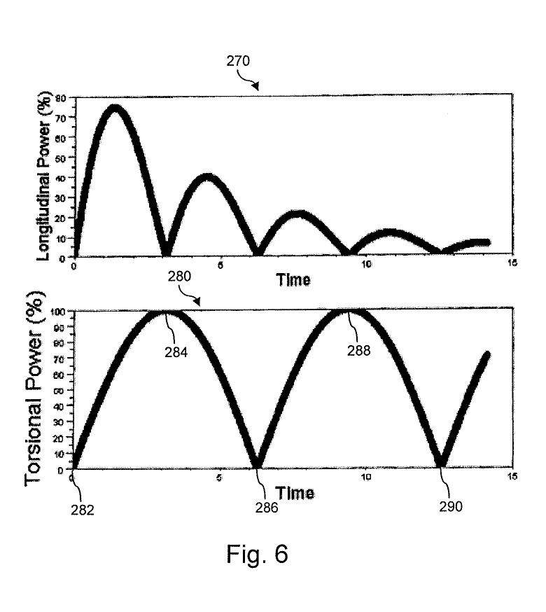

Referring now to FIG. 6, shown therein are a pair of graphs 270 and 280 illustrating longitudinal power versus time and torsional power versus time, respectively, according to another embodiment of the present disclosure. In that regard, if desired a user can control ultrasound longitudinal power and ultrasound torsional power separately. The graphs 270 and 280 illustrate one example of this separate control. In that regard, graph 270 represents longitudinal power versus time, while graph 280 represents torsional power versus time. The longitudinal power versus time mapped in graph 270 is substantially identical to the ultrasound power mapped in graph 230 discussed above with respect to FIG. 4 and, therefore, will not be described in detail here. As shown in graph 280, the torsional power varies non-linearly with respect to time. In that regard, the torsional power repeatedly increases and decreases with time where the corresponding maximum power of each increase in power is maintained with each cycle. As shown, the torsional power starts at point 282 with a value of zero, increases to peak 284 (where it reaches approximately 100% of maximum torsional power), decreases back to zero at point 286, increases to peak 288 (where it again reaches approximately 100% of maximum power), decreases back to zero at point 290, and increases from there to where the graph 280 stops. When implemented during a surgical procedure, this ultrasound power profile will cause the hand piece tip to be displaced in accordance with the combined effects of the longitudinal and torsional power profiles.

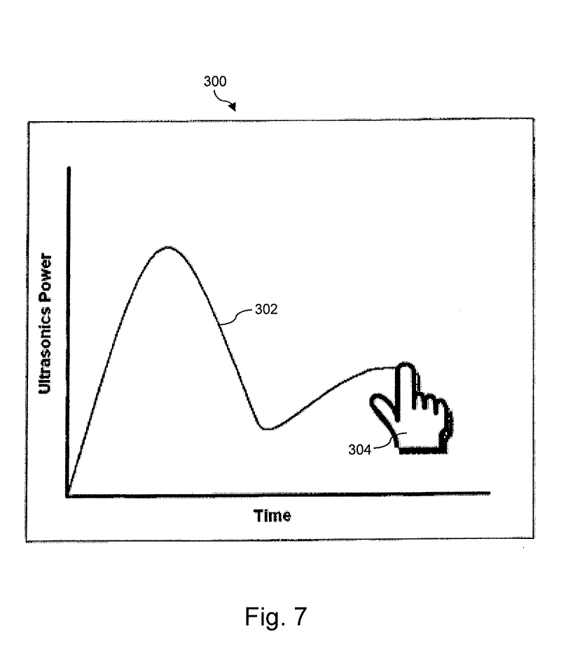

Referring generally to FIGS. 7-17, exemplary ways in which the ultrasound power profiles and/or other operating parameters are defined and/or adjusted will be discussed.

Referring more specifically to FIG. 7, shown therein is a portion of an interactive graphical user interface (“GUI”) 300 that allows a user to define ultrasound power versus time using a freeform input according to one embodiment of the present disclosure. As shown, the ultrasound power increases and decreases in a non-linear fashion as defined by graph line 302.

In that regard, the contours of graph line 302 are defined by the user's freeform input. For example, in some instances the user draws the graph line 302 by moving a finger or stylus along the touch screen of the console. Alternatively, the user draws the graph line 302 by moving a mouse, track ball, or other input device such that an icon on the GUI 300 defines the path of graph line 302. In that regard, hand 304 is intended to represent inputs through the touch screen, using an input device separate from the touch screen, and/or combinations thereof.

The freeform input allows the user great freedom in defining the values or characteristics of the various operating parameters of the surgical console and associated subsystems. In that regard, generally there may be as many unique line segments as the resolution of the display screen and input device (understood to include the touch screen and/or a separate input device) allows. In some instances, the user interface allows the user to zoom in on a particular parameter in order to increase the level of detail visible to the user. In that regard, while a particular operating parameter field may appear to the user to have a continuous drawing area, each operating parameter necessarily has maximum resolution or level of detail to which the system will control the operating parameter within the range of available values for that operating parameter. In that regard, the Chart 1 below illustrates exemplary ranges and resolutions for operating parameters of an ophthalmic surgical console that allows freeform input of the values or characteristics of the operating parameters by a user.

CHART 1

| Mechanism | Operating Parameter | Range | Resolution |
|---|---|---|---|
| Fluidics | Flow Rate | 0 . . . 100 CCPM | 0.25 CCPM |
| | Vacuum Level | 0 . . . 700 mmHg | 1 mmHg |
| Ultrasonics | Power/Amplitude | 0 . . . 100% | 1% |
| | Level | | |
| | Power/Amplitude | 0.25 ms . . . Constant | 0.25 ms |
| | Change Rate | | |
| | On/Off Time | 0 . . . 2500 ms | 0.25 ms |
| | Pulses Per Second | 1 . . . 250 PPS | 1 PPS |
| | Duty Cycle | 0 . . . 100% | 0.25% |
| Vit Cutter | Cut Rate | 6 . . . 5000 CPM | 1 CPM |
| | Duty Cycle | 25 . . . 75% | 0.25% |
| Coagulator | Power Level | 0 . . . 100% | 1% |
| IV Pole | Pole Travel | 0 . . . 100 cm | 1 cm |

It is understood that the ranges and resolutions provided in Chart 1 are provided merely for example and should not be considered limiting. In that regard, it is appreciated that actual ranges and resolutions will vary from system to system. For example, for the operating parameters in Chart 1 whose ranges are not defined by a percentage, it is understood that the upper and lower limits of the range may be increased or decreased by a factor of 10 or more, in some instances. For the operating parameters in Chart 1 whose ranges are defined by a percentage, in some embodiments the range can extend from 0% to 100% or any subset of percentages therebetween. Further, the resolutions of any of the operating parameters in Chart 1 may similarly be increased or decreased by a factor of 10 or more, in some instances. Further, it is understood that any subsets of values within the ranges and resolution described above (including the increases and decreases by a factor of 10 or more) are within the scope of the present disclosure.

Thus, while a user may draw what appears to be a continuous line defining a particular operating parameter, it is understood that the “continuous” line is really a plurality of interconnected set points. In that regard, where there are gaps or holes in a user's “continuous” line, the system will interpolate between the set points defined by the user's line to fill in the gaps or holes. As discussed below, the system may interpolate between set points linearly, using smooth curve or best fit algorithm, and/or combinations thereof. Accordingly, a set point is generally understood to mean a value or characteristic of an operating parameter set by the user. Typically, set points will be visually represented on the display in some manner, such as by a particular icon or as part of a freeform input (such as graph line 302).

The ability to provide these types of customized controls can be useful for improving patient outcomes, user satisfaction with the console, and safety. As one example, the customized controls can allow the user to provide for cooling or fluid flow increase proportional to the ultrasound power being delivered. Accordingly, the customized control can reduce instances of excessive ultrasound power with insufficient irrigation and aspiration that can cause severe damage to the surrounding tissue.

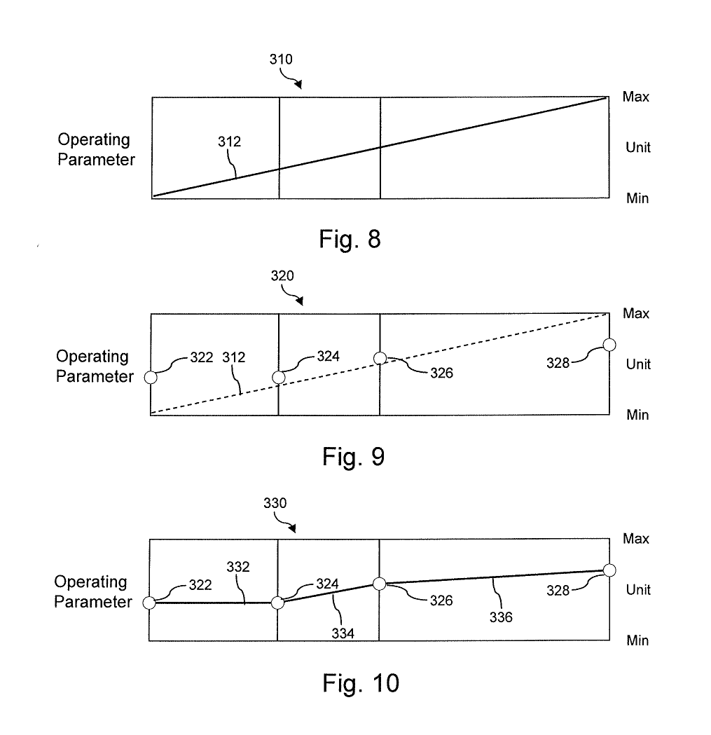

Referring now to FIGS. 8-10, shown therein is an adjustment of an operating parameter using linear interpolation according to one embodiment of the present disclosure. In that regard, FIG. 8 illustrates a baseline or original graphical representation for an operating parameter; FIG. 9 illustrates a plurality of user-selected set points relative to the original graphical representation of the operating parameter shown in FIG. 8; and FIG. 10 illustrates a modified graphical representation of the operating parameter in accordance with the user-selected set points of FIG. 9.

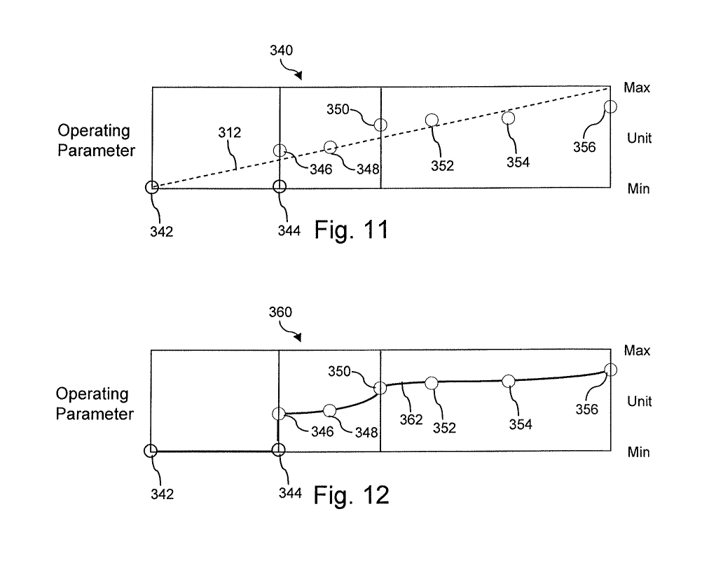

Referring more specifically to FIG. 8, shown therein is a graph 310 illustrating a baseline or original graphical representation 312 for an operating parameter. It is understood that the operating parameter may be any operating parameter of an ophthalmic surgical console or associated subsystem as discussed throughout the present disclosure. As shown, the original graphical representation 312 of the operating parameter defines a linear increase in the value of the operating parameter from the minimum value of the operating parameter (at the bottom left corner corresponding to very beginning of a controller actuation, such as a foot pedal depression) to the maximum value of the operating parameter (at the top right corner, corresponding to the end of a controller actuation, such as full depression of the foot pedal). This baseline or original graphical representation 312 of the operating parameter will be used for subsequent descriptions below with respect to FIGS. 11-16, as well as FIGS. 9 and 10.

Referring now to FIG. 9, shown therein is a graph 320 illustrating a plurality of user-defined or user-selected set points 322, 324, 326, and 328 relative to the original graphical representation 312 for the operating parameter. The set points represent user-desired changes in the value of the operating parameter at the corresponding controller position. In that regard, the set points may be defined or selected by the user in a number of ways. In some instances, the user simply touches the screen (with a finger, stylus, or other object) at the desired locations of the set points. In other instances, the user manipulates an input device separate from the touch screen to identify the desired locations. For example, in some embodiments the user will double click a mouse at the location of a set point. In other instances, the user will slide or move a portion of the visual representation of the operating parameter (e.g., by dragging a finger, stylus, or other object) to a desired location representing the set point. The user may similarly slide or move a portion of the visual representation using an input device separate from the touch screen (e.g., using the click and drag function of a mouse). In some instances, the user will define the value of an operating parameter at a particular location (thereby defining a set point) by typing the desired value into a keyboard. The keyboard may be a part of the user interface or display or the keyboard may be separate from the display.

As discussed below, selecting or defining the set points will cause the graphical representation of the operating parameter to adjust to match the set points. In that regard, in some instances the adjustments are made in approximately real time (i.e., as the system processes the inputs from the user) with each set point input. In other instances, the adjustments are not made until the user provides a command to make the adjustments, which may be after one or more set point adjustments, including after all set point adjustments have been made.

Referring now to FIG. 10, shown therein is a graph 330 illustrating a modified graphical representation of the operating parameter in accordance with the user-selected set points 322, 324, 326, and 328. In that regard, line segment 332 extends between set points 322 and 324, line segment 334 extends between set points 324 and 326, and line segment 336 extends between set points 326 and 328. The line segments 332, 334, and 336 were defined by linearly interpolating between the user-selected set points 322, 324, 326, and 328. That is, a straight line extends between each of the adjacent set points. In contrast to the constant linear increase in the operating parameter defined by the original graphical representation 312, the modified graphical representation shown in graph 330 is variable across the foot pedal ranges (consistent with FIG. 3 discussed above, the different stages are separated by vertical line dividers). More specifically, line segment 332 defines a constant value for the operating parameter during one of the ranges. Line segment 334 defines a linear increase in the operating parameter within another range. Finally, line segment 336 also defines a linear increase in the operating parameter during the third foot pedal range, but at a lower rate of increase than that defined by line segment 334.

Referring now to FIGS. 11 and 12, shown therein is an adjustment of an operating parameter using a smooth curve or best fit interpolation according to one embodiment of the present disclosure. Referring more specifically to FIG. 11, shown therein is a graph 320 that illustrates a plurality of user-selected set points 342, 344, 346, 348, 350, 352, 354, and 356 relative to the original graphical representation 312 of the operating parameter shown in FIG. 8. In that regard, the set points 342, 344, 346, 348, 350, 352, 354, and 356 can be defined in any manner contemplated by the present disclosure. It should be noted that set points 342 and 344 are included simply for clarification that the operating parameter should have the minimum value through the first stage of the operation. In some embodiments, the system will be programmed to recognize that the operating parameter is not utilized during one or more stages of an operation and, therefore, should either not be activated and/or have the minimum value during that stage or stages of the operation. Accordingly, in some instances set points 342 and 344 are omitted. In that regard, the user is able to delete the existing segment of graphical representation 312 shown within the first stage.

Referring now to FIG. 12, shown therein is a graph 360 illustrating a modified graphical representation 362 of the operating parameter in accordance with the user-selected set points 342, 344, 346, 348, 350, 352, 354, and 356. In that regard, the graphical representation 362 is a smooth curve mapping between the user-selected set points 342, 344, 346, 348, 350, 352, 354, and 356. However, it is understood that any type of best fit algorithm may be utilized to define the graphical representation of the operation parameter based on the user-selected set points 342, 344, 346, 348, 350, 352, 354, and 356. Generally, the graphical representation 362 of the operating parameter indicates that the operating parameter will increase through the second and third stages of the operation at varying rates.

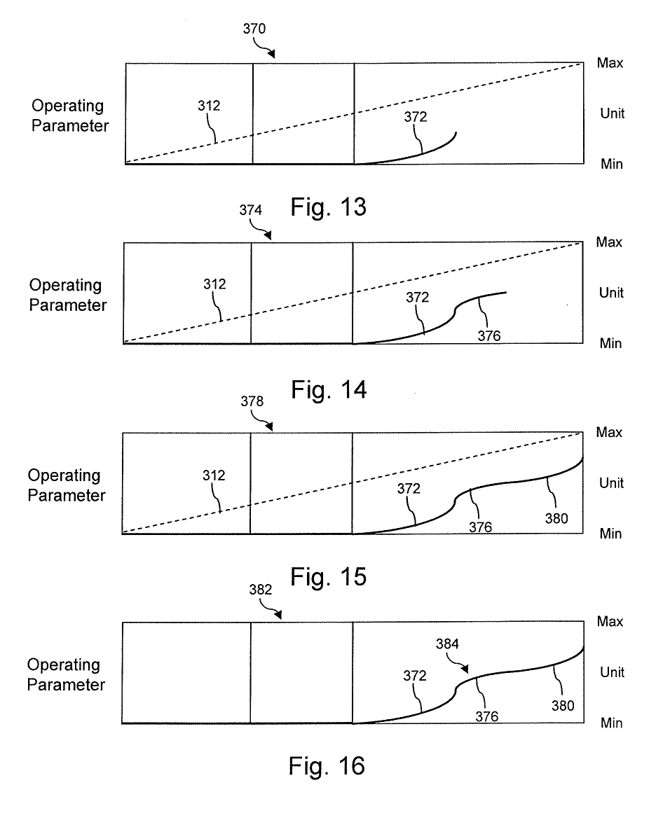

Referring now to FIGS. 13-16, shown therein is an adjustment of an operating parameter using a freeform user input according to one embodiment of the present disclosure. In that regard, referring more specifically to FIG. 13, shown therein is a graph 370 that illustrates a first portion 372 of a freeform user input relative to the original graphical representation 312 of the operating parameter shown in FIG. 8. Referring now to FIG. 14, shown therein is a graph 374 that illustrates a second portion 376 of the freeform user input along with the first portion 372 relative to the original graphical representation 312 of the operating parameter. Similarly, FIG. 15 provides a graph 378 that illustrates a third portion 380 of the freeform user input along with the first and second portions 372 and 376 relative to the original graphical representation 312 of the operating parameter. Finally, referring to FIG. 16, shown therein is a graph 382 that illustrates a modified graphical representation 384 of the operating parameter in accordance with the freeform user inputs 372, 376, and 380 of FIGS. 13-15. It is understood that the freeform user inputs 372, 376, and 380 can be defined by the user in any manner or input mechanism contemplated by the present disclosure.

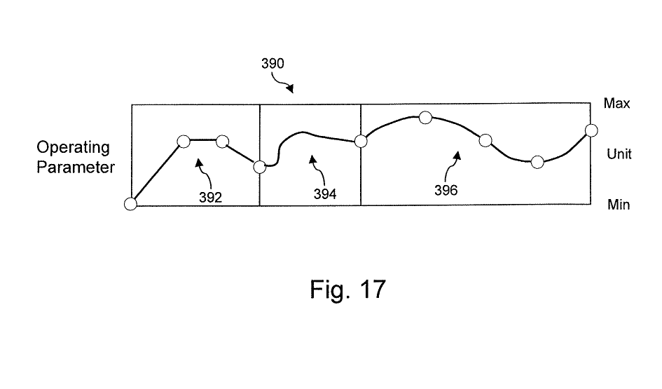

Referring now to FIG. 17, shown therein is graph 390 that illustrates adjustment of an operating parameter using a combination of linear interpolation, smooth curve interpolation, and freeform user input according to one embodiment of the present disclosure. As shown, the operating parameter has been defined by a linear interpolation for the first stage of the procedure as indicated by graphical representation 392. For the second stage of the procedure, the operating parameter has been defined by a freeform user input as indicated by graphical representation 394. Finally, for the third stage of the procedure, the operating parameter has been defined by a smooth curve or best fit interpolation as indicated by graphical representation 396.

It is understood that the portions of the graphical user interfaces depicted in the accompanying drawings and described above are not exhaustive or all-inclusive of the operating parameters, characteristics, values, or otherwise that will displayed to the user on the screen. Rather, the portions of the graphical user interfaces of the present disclosure are intended to be used in combination with numerous display features, including without limitation other operating parameters, characteristics, values, and information that may be displayed to a user in the context of an ophthalmic procedure. For example, it is specifically noted that real time values of one or more of the various operating parameters are displayed to the user in some embodiments.

Further, it is understood that the depictions of the operating parameter graphs, stage dividers, and other visual aspects of the exemplary embodiments are for the purposes of illustration, not limitation. It is fully contemplated that these features can be displayed in a wide variety of alternative ways and combinations, including various types of graphs, orientations, shapes, colors, etc.

Also, it is understood that while the graphical user interfaces and associated functionality have been described as being part of the ophthalmic surgical console 100 and, in some respects, particularly part of the computer system 103, in some instances the graphical user interface runs on a computing device (including handheld devices) separate from the surgical console 100. In that regard, the computing device is in communication with the surgical console 100 (wirelessly, wired, or through other means such as a memory storage device) such that the control provided to the user by the graphical user interfaces of the present disclosure is still imparted to the surgical console and associated subsystems.

In some embodiments, a user may save or store particular operating profiles for use in later procedures. In that regard, the graphical user interface will allow the user to select from a set of pre-programmed profiles or previously saved profiles. The stored profiles may relate to an entire procedure, multiple stages of a procedure, and/or a single stage of a procedure. Further, the profiles may relate to multiple operating parameters and/or a single operating parameter for the entire procedure, multiple stages of the procedure, and/or a single stage of the procedure. By allowing a user to define the operating parameters for the various profiles and then subsequently select from a variety of preprogrammed or saved profile options, the user is able to tailor the operating parameters to the characteristics of a particular patient and/or the user's preferences (e.g., where the console is be used by multiple users).

Persons skilled in the art will appreciate that the linear and non-linear representations of the operating parameters shown on the display may not correlate precisely with the actual output or measurement of that operating parameter. This may be due to a variety of factors including, but not limited to tolerances, resolutions, limits, or other factors within or associated with the console and/or associated subsystems. For example, where the ultrasound power is defined by a linear function, the actual relationship between the power and the position of the foot pedal may not be exactly linear due to mapping the foot pedal position to the amount of power that is generated. Thus, there may be some deviations from a truly “linear” representation in practice due to mapping and other factors. It is understood that in the context of the present disclosure these variances or deviations between the visual representations of the operating parameters and the actual outputs or measurements of the operating parameters should still be considered to be part of the operating parameter as defined by the visual representations.

In some instances, the console 100 will limit the user's ability to vary the operating parameters and/or automatically adjust the operating parameters. This may be due to factors such as operating limitations of the console and/or subsystems, patient safety, and/or other factors. For example, in some embodiments one operating parameter may be linked to another operating parameter such that the system ensures that the settings for each of the parameters are proper relative to the settings for the other parameters.

Accordingly, in some instances the system will prevent adjustment of an operating parameter outside of a certain range. In other instances, the system will adjust the linked parameter to accommodate for changes in the other linked parameter. In some embodiments the system provides a notification to the user of the action taken (limiting the adjustment or adjusting a linked parameter) to coordinate the parameters.

Persons skilled in the art will also recognize that the graphical user interface and adjustments to the operating parameters can be modified in various ways. Accordingly, persons of ordinary skill in the art will appreciate that the embodiments encompassed by the present disclosure are not limited to the particular exemplary embodiments described above. In that regard, although illustrative embodiments have been shown and described, a wide range of modification, change, and substitution is contemplated in the foregoing disclosure. It is understood that such variations may be made to the foregoing without departing from the scope of the present disclosure. Accordingly, it is appropriate that the appended claims be construed broadly and in a manner consistent with the present disclosure

What is claimed is:

1. An ophthalmic surgical console system, comprising:
an ultrasound generator;
a hand piece in communication with the ultrasound generator such that the hand piece receives ultrasound signals from the ultrasound generator and a tip of the hand piece is actuated in response to the received ultrasound signals;
a display configured to display an interactive graphical user interface that includes a visualization of an ultrasound operating parameter, wherein the interactive graphical user interface is configured to receive a freeform user input that defines at least a portion of the ultrasound operating parameter;
a computing device in communication with the ultrasound generator and the display, the computing device configured to communicate the ultrasound operating parameter to the ultrasound generator such that the ultrasound signals received by the hand piece from the ultrasound generator are based upon the ultrasound operating parameter as defined by the freeform user input.

2. The system of claim 1, wherein the display is a touch screen and the interactive graphical user interface receives the freeform user input via the touch screen.

3. The system of claim 2, wherein the freeform user input is a drawing of a function of the ultrasound operating parameter relative to positions of a controller that is in communication with the computing device.

4. The system of claim 3, wherein the controller is a foot pedal.

5. The system of claim 4, wherein the ultrasound operating parameter is selected from the group consisting of an ultrasound power level, an ultrasound on-time, an ultrasound off-time, ultrasound pulses per second, and an ultrasound duty cycle.

6. The system of claim 5, wherein the interactive graphical user interface includes visualizations of a plurality of non-ultrasound operating parameters, wherein the interactive graphical user interface is configured to receive user inputs that define at least a portion of each of the plurality of non-ultrasound operating parameters.

7. The system of claim 6, wherein the plurality of non-ultrasound operating parameters include one or more of an intravenous pole height, an aspiration flow rate, a vacuum pressure level, an vitreous cutter cut rate, a vitreous cutter duty cycle, and a coagulator power level.

8. The system of claim 7, wherein the interactive graphical user interface is configured to receive a plurality of set points from the user via the touch screen to define at least a portion of each of the plurality of non-ultrasound operating parameters.

9. The system of claim 8, wherein the interactive graphical user interface is configured to adjust the visualizations of the plurality of non-ultrasound operating parameters based on the plurality of set points received from the user.

10. The system of claim 9, wherein the computing device controls the interactive graphical user interface.

11. A surgical console system, comprising:
a computer system;
a touch screen display in communication with the computer system;
a controller in communication with the computer system, the controller movable between a plurality of positions; and
a surgical device in communication with the computer system such that the surgical device receives operational signals from the computer system, the surgical device operating in accordance with the operational signals received from the computer system;
wherein the computer system is configured to:
output an interactive graphical user interface signal to the touch screen display such that the touch screen display displays the interactive graphical user interface;
receive a first freeform user input via the touch screen displaying the interactive graphical user interface, the first freeform user input defining values of a first operational parameter of the surgical device in relation to the plurality of positions of the controller; and
send the operational signals to the surgical device based on the position of the controller and the values of the first operational parameter as defined by the first freeform user input.

12. The system of claim 11, wherein the surgical device is an ultrasound hand piece.

13. The system of claim 12, wherein the first operating parameter is longitudinal ultrasound power.

14. The system of claim 13, wherein the computer system is further configured to:
receive a second user input via the touch screen displaying the interactive graphical user interface, the second user input defining values of a second operational parameter of the surgical device in relation to the plurality of positions of the controller; and
send the operational signals to the surgical device based on the position of the controller and the values of the first and second operational parameters as defined by the first freeform user input and the second user input.

15. The system of claim 14, wherein the second operating parameter is a torsional ultrasound power.

16. The system of claim 11, wherein the surgical device is a vitreous cutter.

17. The system of claim 16, the first operating parameter is selected from the group consisting of vitreous cut rate and vitreous duty cycle.

18. An ophthalmic surgical method, comprising:
receiving, via an interactive graphical user interface, a freeform input from a user of an ophthalmic surgical system, the freeform input defining a characteristic of an operating parameter of the ophthalmic surgical console;
operating the ophthalmic surgical console such that the operating parameter is controlled in accordance with the freeform input received from the user via the interactive graphical user interface.

19. The method of claim 18, wherein the freeform user input received from the user via the interactive graphical user interface is a drawing of a function of the operating parameter of the ophthalmic surgical system relative to positions of a controller of the ophthalmic surgical system.

20. The method of claim 19, wherein the operating parameter is selected from the group consisting of intravenous pole height, aspiration flow rate, vacuum limit pressure, ultrasound power, ultrasound on-time, and ultrasound off-time.
